(19) United States
(12) Patent Application Publication
(10) Pub. No.: US 2006/0259020 A1
(43) Pub. Date: Nov. 16, 2006

(54) Bacteria resistant coating for surgical instrument

(75) Inventor: Todd W. Sharratt, Stillwater, MN (US)

Correspondence Address: WESTMAN CHAMPLIN & KELLY, P.A., SUITE 1400, 900 SECOND AVENUE SOUTH, MINNEAPOLIS, MN, 55402-3319, US

(73) Assignee: Minnesota Scientific, Inc., St. Paul, MN (US)

(21) Appl. No.: 11/443,482

(22) Filed: May 30, 2006

Related U.S. Application Data
(63) Continuation-in-part of application No. 10/942,182, filed on Sep. 16, 2004.
(60) Provisional application No. 60/503,642, filed on Sep. 17, 2003.

(51) Int. Cl. A61B 17/00

(52) U.S. Cl. 606/1

(57) ABSTRACT

A surgical instrument for use in a surgical site includes a first surface that is positionable within or near the surgical site and has an anti-bacterial coating disposed on the surface. The anti-microbial coating includes anti-microbial particles disposed in a polymer matrix wherein the anti-microbial particles are in sufficient concentration and are positioned to provide an anti-microbial effect at the surgical site. Bacterial growth is also inhibited on the coated surface of the instrument.

### CROSS-REFERENCE TO RELATED APPLICATION

The present application is a continuation-in-part of and claims priority of U.S. patent application Ser. No. 10/942,182, filed Sep. 16, 2004, the content of which is hereby incorporated by reference in its entirely.

The present application claims priority of U.S. Provisional Application Ser. No. 60/503,642, filed Sep. 17, 2003, the content of which is hereby incorporated by reference in its entirety.

### BACKGROUND OF THE INVENTION

This invention relates to anti-microbial coatings on surgical instruments and a method of reducing bacterial growth on surgical instruments.

Currently, in the United States alone, an estimated 27 million surgical procedures are performed each year. Centers for Disease Control and Prevention, National Center for Health Statistics. Vital and Health Statistics, Detailed Diagnoses and Procedures, National Hospital Discharge Survey, 1994. Vol. 127. Hyattsville, Md.: VHHS Publication; 1997. Based on the Center for Disease Control monitoring of infections in U.S. hospitals, surgical site infections (SSIs) of the third most frequently reported nosocomial infection accounting for about 14 to 16% of all nosocomial infections among hospitalized patients. Emori T. G., Gaynes R. P., An Overview of Nosocomial Infections, including the Role of the Microbiology Laboratory. Clin Microbiol Rev. 1993; 6(4); 428-42. It has also been reported that approximately two-thirds of SSIs were confined to the incision while approximately one-third of the SSIs involve organs or spaces accessed during the surgery.

There have been advances in infection control practices including improved operating room ventilation, sterilization methods, barriers, surgical technique, and availability of anti-microbial prophylaxis. Despite these activities, SSIs remain a substantial cause of morbidity and mortality among hospitalized patients. Mangram A. J., Horn T. C., Pearson, M. L., Silver, L. C., Jarvis, W. R., Guideline for prevention of Surgical Site Infection, 1999, Infection Control and Hospital Epidemiology, V. 20 No. 4, 246-78.

A number of metal ions have been shown to possess antibiotic activity, including silver, copper, zinc, mercury, tin, lead, bismutin, cadmium, chromium and thallium ions. It is theorized that these antibiotic metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver ions are particularly useful for in vivo uses due to the fact that they are not substantially absorbed into the body.

Silver ions have been impregnated in the surfaces of medical implants, as described in U.S. Pat. No. 5,474,797. Silver ions have also been incorporated in catheters, as described in U.S. Pat. No. 5,520,664. The products described in these patents, however, do not exhibit an antibiotic effect for a prolonged period of time because a passivation layer typically forms on the silver ion coating. This layer reduces the release rate of the silver ions from the product, resulting in lower antibiotic effectiveness. In addition, the layer containing the silver frequently becomes discolored, causing the products to have a poor appearance. The discoloration is caused by a high flux release rate of silver ion into the surroundings.

Antibiotic zeolites can be prepared by replacing all or part of the ion-exchangeable ions in zeolite with antibiotic metal ions, as described in U.S. Pat. Nos. 4,011,898; 4,938,955; 4,906,464; and 4,775,585. Polymers incorporating antibiotic zeolites have been used to make refrigerators, dish washers, rice cookers, plastic film, chopping boards, vacuum bottles, plastic pails, and garbage containers. Other materials in which antibiotic zeolites have been incorporated include flooring, wallpaper, cloth, paint, napkins, plastic automobile parts, bicycles, pens, toys, sand, and concrete. Examples of such uses are described in U.S. Pat. Nos. 5,714,445; 5,697,203; 5,562,872; 5,180,585; 5,714,430; and 5,102,401.

Hydrophilic coatings with low friction have been applied to medical devices such as catheters. See, for example, U.S. Pat. No. 5,509,899. Such coatings are highly desirable to allow for easy insertion into the body. Hydrophilic coatings, however, are excellent breeding grounds for bacteria.

U.S. Pat. No. 4,923,450 discloses a catheter having a coating of antibiotic zeolite. U.S. Pat. No. 5,100,671 describes a medical article that is formed using silicone rubber that contains antibiotic zeolite. However, use of conventional antibiotic zeolite, such as that described in U.S. Pat. No. 4,011,898, results in a catheter which exhibits severe discoloration. For example, a catheter made according to U.S. Pat. No. 4,923,450 which has a coating of the antibiotic zeolite material of U.S. Pat. No. 4,011,898 adhered to its surface becomes highly discolored within days.

A conventional catheter is typically comprised of a hydrophobic polymer. When antibiotic zeolite is incorporated in such a catheter, however, water is unable to reach the zeolite in the bulk of the material. The bulk of the zeolite is, therefore, ineffective against bacteria surrounding the catheter since only the zeolite at the surface of the catheter is active.

U.S. Pat. No. 5,305,827 describes an anti-microbial hydrophilic coating for heat exchangers. The coating includes silver oxide, to inhibit microbial growth and improved adhesion to the heat transfer surfaces of a heat exchanger. However, this coating exhibits severe discoloration and is typically anti-microbially effective for 3 days or less.

Japanese Pat. Application No. 03347710 relates to a non-woven fabric bandage containing synthetic fibers and hydrophilic fibers. The synthetic fibers contain zeolite which is ion-exchanged with silver, copper, or zinc ions.

U.S. Pat. No. 4,923,450 discloses incorporating zeolite in bulk materials. When zeolite is conventionally compounded into polymers, however, the zeolite often aggregates, causing poor dispersion of the zeolite in the polymer. When such material is molded or extruded, the surface of the polymer is frequently beaded instead of flat. Poor dispersion of the zeolite also can cause changes in the bulk properties of the polymer, such as a reduction in tensile strength. Any significant changes in the bulk properties of medical devices, such as catheters, however, result in a need to seek regulatory clearance by the U.S. Food and Drug Administration (FDA), which is a costly and time consuming process.

Furthermore, it has been found that conventionally kneading antibiotic zeolites in many polymeric materials results in a “hazy” appearance and in discoloration. This appears also to result from inadequate dispersion of the zeolite, for example, the formation of zeolite aggregates in the material, and the inclusion of air or water during the kneading process.

U.S. Pat. No. 4,938,958 describes antibiotic zeolites in which a portion of the ion-exchangeable ions in the zeolite are replaced with ammonium. This results in a product which exhibits reduced discoloration. However, as described in U.S. Pat. No. 4,938,955, it is often necessary to add an organic discoloration inhibitor, in addition to the antibiotic zeolite, to adequately prevent discoloration of the resin in which the zeolite is incorporated. Discoloration inhibitors are often not biocompatible and cannot be incorporated into medical devices. Furthermore, incorporation of an organic discoloration inhibitor in the polymeric material of a medical device may cause changes in the bulk properties of the material that are highly undesirable.

All patent applications, patents, patent publications, and literature references cited in this application are hereby incorporated by reference in their entirety. In the case of inconsistencies, the present application, including definitions, is intended to control.

### SUMMARY OF THE INVENTION

The present invention includes a surgical instrument for use in a surgical site. The surgical instrument includes a surgical surface that is positioned within the surgical site and an anti-bacterial coating disposed on the surgical surface. The anti-microbial coating includes anti-microbial particles disposed in a polymeric matrix wherein the anti-microbial particles are in sufficient concentration and are positioned within the matrix to provide an anti-microbial effect at the surgical site.

The present invention also includes a method of inhibiting bacterial growth on surfaces of surgical instruments that are used in surgery. The method includes providing an anti-bacterial coating on at least the surgical surface of a surgical instrument. The surgical surface is that surface that is inserted into a surgical site. The anti-microbial coating includes anti-microbial particles disposed in a polymeric matrix wherein the anti-microbial particles are in sufficient concentration and are positioned within the matrix to provide an anti-microbial effect at the surgical site.

The present invention also includes a method of reducing infection at a surgical site or comes in contact or near contact of the surgical site. The method includes utilizing a surgical instrument having an anti-microbial coating on surgical surfaces that are inserted into the surgical site with the anti-microbial coating comprising anti-microbial particles dispersed within a polymer matrix wherein the anti-microbial particles are in sufficient concentration and are positioned in the matrix to provide an anti-microbial effect at the surgical site.

### BRIEF DESCRIPTION OF THE DRAWINGS

FIG. 1 is a perspective view of a surgical retractor with an anti-microbial coating of the present invention.

### DETAILED DESCRIPTION OF ILLUSTRATIVE EMBODIMENTS

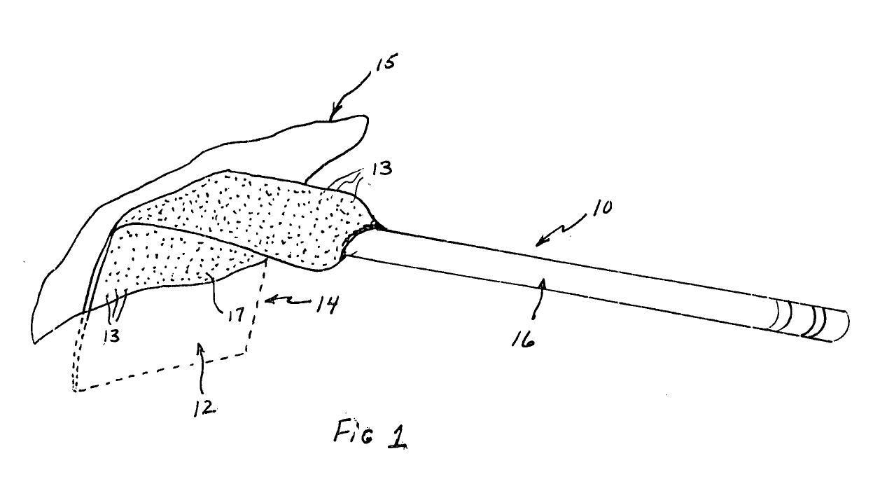

A surgical retractor generally indicated at 10 in FIG. 1 includes an anti-microbial coating 12 of the present invention on blade 14. The anti-microbial coating includes anti-microbial particles 13 within an autoclavable polymeric matrix 17. The surgical retractor further includes a handle 16 to which the blade 14 is attached. The blade 14 is that portion of the surgical retractor that is inserted into an incision site 15 for retaining tissue to provide the surgeon access for performing the surgery.

Infections are a reoccurring problem during surgery. Retractors and other surgical instruments used in surgery are, of course, sterilized to reduce or eliminate bacterial infection. However, infections still occur occasionally. Whether the cause or source of such infections are the surgical instruments or other factors is not known. The anti-microbial coating of the present invention is intended to eliminate, reduce or inhibit bacterial growth on surfaces of the surgical instrument thereby removing a source of bacterial infection. Also the anti-microbial coating of the present invention provides a positive factor to reduce bacterial count that may occur from other sources at the surgical site.

By surgical instruments is meant not only surgical retractors but also forceps, surgical racks, bone hooks, scalpels, and other surgical knifes, scissors, tracheal dilators and tracheal tubes, surgical probes, speculums, surgical depressors and dilators, syringes, spatulas, endoscopes, arthroscopes and any other instruments that have a surface that is inserted into the surgical site.

By surgical site is meant the incision or wound that is made in the patient in typically incising the skin, subcutaneous and deep soft tissue (fascia and muscle) to typically reach an organ or skeletal element for need of repair or replacement. Incision site within this definition includes those open incisions and those referred to as closed wounds or incisions in which endoscopic or arthroscopic surgery is performed.

The surfaces of the surgical retractor or other surgical instruments of the present invention that are positioned within the incision are coated with the anti-microbial coating of the present invention. The anti-microbial coating contains an inorganic anti-microbial agent. The anti-microbial agent is disposed in a polymeric matrix which adheres the anti-microbial agent to the surfaces of the surgical retractor or other surgical instrument and presents the anti-microbial agent in a manner to be effective against pathogens.

One polymer useful in the present invention is polyetheretherketone (PEEK). Other polymeric materials that are suitable candidates include liquid crystal polymer, mineral filled nylon, polyarylate, polyethermide, polyethersulfane, polyester, polyphenylene sulfide and polysulfone. Other plastics that can withstand sterilization (autoclaving) temperatures and are approved by the appropriate governmental agencies for use in surgery are also included within the present invention.

The inorganic anti-microbial agent is useful in a form of an antibiotic ceramic particle. Antibiotic ceramic particles include, but are not limited to, zeolites, hydroxyapatite, zirconium phosphates and other ion-exchange ceramics. Hydroxyapatite particles containing antimicrobial metals are described, for example, in U.S. Pat. No. 5,009,898. Zirconium phosphates containing antimicrobial metals are described, for example, in U.S. Pat. Nos. 5,296,238; 5,441,717; and 5,405,644. One useful ceramic particle is an antibiotic zeolite containing ion-exchanged antibiotic metal ions.

The coating utilizing the antiobotic zeolite has a thickness ranging from about 0.004 inch (0.1 mm) 0.050 inches (1.30 mm). Thickness ranging up to 5 mm are also within the present invention. As will be appreciated by those skilled in the art, however, the optimal thickness of coating employed will depend on the substrate being coated. Typically the substrate being coated is metallic but plastic and composite substrates are also contemplated.

An amount of antibiotic ceramic is dispersed in the polymer that is effective to release the antibiotic metal ions in a microbiocidal effective amount. A release rate ranging from about 5 to about 50 ppb of microbiocidally effective silver ions upon contact with body tissues has been found to be effective.

A number of metal ions, which are inorganic materials, have been shown to possess antimicrobial activity, including silver, copper, zinc, mercury, tin, lead, bismuth, cadmium, chromium and thallium ions. These antimicrobial metal ions are believed to exert their effects by disrupting respiration and electron transport systems upon absorption into bacterial or fungal cells. Antimicrobial metal ions of silver, gold, copper and zinc, in particular, are considered safe even for in vivo use. Antimicrobial silver ions are particularly useful for in vivo use due to the fact that they are not substantially absorbed into the body. That is, if such materials are used they should pose no hazard.

In one embodiment of the invention, the inorganic antimicrobial metal containing composition is an antimicrobial metal salt. Such salts include silver acetate, silver benzoate, silver carbonate, silver ionate, silver iodide, silver lactate, silver laureate, silver nitrate, silver oxide, silver palpitate, silver protein, and silver sulfadiazine. Silver nitrate is preferred. These salts are particularly quick acting, so no release from ceramic particles is necessary to function antimicrobially.

Antimicrobial zeolites are preferred. These have been prepared by replacing all or part of the ion-exchangeable ions in zeolite with ammonium ions and antimicrobial metal ions, as described in U.S. Pat. Nos. 4,938,958 and 4,911,898. Such zeolites have been incorporated in antimicrobial resins (as shown in U.S. Pat. Nos. 4,938,955 and 4,906,464) and polymer articles (U.S. Pat. No. 4,775,585). Polymers including the antimicrobial zeolites have been used to make refrigerators, dish washers, rice cookers, plastic film, chopping boards, vacuum bottles, plastic pails, and garbage containers. Other materials in which antimicrobial zeolites have been incorporated include flooring, wall paper, cloth, paint, napkins, plastic automobile parts, catheters, bicycles, pens, toys, sand, and concrete. Examples of such uses are described in U.S. Pat. Nos. 5,714,445; 5,697,203; 5,562,872; 5,180,585; 5,714,430; and 5,102,401.

Inorganic particles, whose core is an oxide of titanium, aluminum, zinc and copper, may be coated with a layer of an antimicrobial metal or metal oxide which confers antimicrobial properties and a protective layer of an alkali metal silicate or aluminate thereby releasing antimicrobial metal ions such as silver ions, are described, for example, in U.S. Pat. No. 5,180,585. Inorganic soluble glass particles containing antimicrobial metal ions, such as silver, are described, for example, in U.S. Pat. Nos. 5,766,611 and 5,290,544.

Antimicrobial zeolites are well-known and can be prepared for use in the present invention using known methods. These include the antimicrobial zeolites disclosed, for example, in U.S. Pat. Nos. 4,938,958 and 4,911,898.

Either natural zeolites or synthetic zeolites can be used to make the antimicrobial zeolites used in the present invention. “Zeolite” is an aluminosilicate having a three dimensional skeletal structure that is represented by the formula: XM_{2/n}O—AL₂O₃—YSiO₂-ZH₂OM represents an ion-exchangeable ion, generally a monovalent or divalent metal ion, n represents the atomic valency of the (metal) ion, X and Y represent coefficients of metal oxide and silica respectively, and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. The present invention is not restricted to use of these specific zeolites.

The ion-exchange capacities of these zeolites are as follows: A-type zeolite=7 meq/g; X-type zeolite=6.4 meq/g; Y-type zeolite=5 meq/g; T-type zeolite=3.4 meq/g; sodalite=11.5 meq/g; mordenite=2.6 meq/g; analcite=5 meq/g; clinoptilolite=2.6 meq/g; chabazite=5 meq/g; and erionite=3.8 meq/g. These ion-exchange capacities are sufficient for the zeolites to undergo ion-exchange with ammonium and antimicrobial metal ions.

The specific surface area of preferred zeolite particles is preferably at least 150 m^{2/g} (anhydrous zeolite as standard) and the SiO₂/Al₂O₃ mol ratio in the zeolite composition is preferably less than 14, more preferably less than 11.

The antimicrobial metal ions used in the antimicrobial zeolites should be retained on the zeolite particles through an ion-exchange reaction. Antimicrobial metal ions which are adsorbed or attached without an ion-exchange reaction exhibit a decreased bactericidal effect and their antimicrobial effect is not long-lasting. Nevertheless, it is advantageous for imparting quick antimicrobial action to maintain a sufficient amount of surface adsorbed metal ion.

During the ion-exchange process, if the concentration of metal ions in the vicinity of the zeolite surface is high, there is a tendency for the antimicrobial metal ions (cations) to be converted into their oxides, hydroxides, basic salts, and the like, which deposit in the micro pores or on the surfaces of the zeolite. This deposition may adversely affect the bactericidal properties of the ion-exchanged zeolite.

One method of applying the antibacterial coating is powder coating. Powder coating techniques are well known in the art. PEEK can be purchased in a form ready for powder coating. The powder coating process usually comprises the basic steps of cleaning the metal surface, electrostatically spraying the polymeric powder and baking. Polymeric powder containing the anti-microbial particle (Agion.™.) manufactured by Agion Technologies, LLC of Wakefield, Mass. is mixed with deionized water to form a paste. The paste is then dried into a powder. The surfaces onto which the powder is applied such as retractor blades or other surgical instruments, are cleaned and then bead blasted. The instruments are hung on a rack and electrostatically charged. The instruments are placed in an oven at a temperature of approximately 700° F. The surfaces are then sprayed with the dried powder forming a coating having a consistent thickness over the surface. The coating is then cooled and reheated again to approximately 700° F. and sprayed with a second coat of the Agion/peek powder. Alternatively, the zeolite particles or the pellets of resin containing the zeolite particles may be applied in a second step to the surface of a part already polymeric powder coated before the baking step. Incorporation of the inorganic antimicrobial into the polymeric powder can be accomplished by preparing a master batch concentrate of pellets containing the antimicrobial particles which is then blended into the same or a different polymer used for the spray coating powder to a selected concentration.

The composite of agent containing antimicrobial particles and the spray polymeric powder is ground or melt atomized to produce a powder that is used directly or diluted with untreated spray powder used in the conventional powder coating process. The powder is applied in the normal manner.

Again, an effective amount of the antimicrobial agent is used. Typically, this is between 0.1 to 30 wt %, preferably 0.5 to 15 wt %, most preferably 1 to 10 wt % of the final powder sprayed on the device.

An alternate method combines untreated polymer powder with a solution of an appropriate solvent, with or without a binder, and adds the inorganic antimicrobial particles to coat the inorganic antimicrobial particles on the polymer powder particles. The solvent is then evaporated and the polymeric powder coated with the antimicrobial agent, is used in the conventional powder coating process. This ensures that the inorganic antimicrobial particle is exposed at the surface of the coating.

Another method of producing an antimicrobial powder coating is to apply a powder coating onto the surface of the retractor blade in the conventional manner and then apply a coating of the inorganic antimicrobial particles in a solvent or water. The retractor blade with coating and antimicrobial particles is then dried and baked as in the conventional powder coating process, thus incorporating the inorganic antimicrobial particles specifically into the near surface of the coating.

Although the present invention has been described with reference to preferred embodiments, workers skilled in the art will recognize that changes may be made in form and detail without departing from the spirit and scope of the invention.

What is claimed is:

1. A surgical instrument for use in a surgical site, the instrument comprising:
a first surface for positioning within the surgical site; and
an anti-bacterial coating comprising anti-microbial particles in a autoclavable polymer matrix wherein the anti-microbial particles are positioned to provide an anti-microbial effect at the surgical site.

2. The surgical instrument of claim 1 wherein the autoclavable polymer matrix comprises a synthetic polymer having sufficient stability to withstand autoclavable temperatures.

3. The surgical instrument of claim 1 wherein the autoclavable polymer matrix comprises polyetheretherketone.

4. The surgical instrument of claim 1 wherein the anti-microbial particles include zeolites, hydroxyapatite and zirconium phosphates.

5. The surgical instrument of claim 1 wherein the anti-microbial particles comprise an antibiotic ceramic comprising antibiotic metal ions.

6. The surgical instrument of claim 5 wherein the antibiotic metal ions comprise silver, copper, zinc, mercury, tin, lead, bismuth, cadmium, chromium and thallium ions.

7. The surgical instrument of claim 1 wherein the anti-microbial particles comprise inorganic anti-microbial metal salts.

8. The surgical instrument of claim 1 wherein the anti-bacterial coating has a thickness from about 0.1 mm to about 5.0 mm.

9. A method for inhibiting bacterial growth on a surgical instrument having a surface positionable in or near a surgical site, the method comprising:
coating the surface of the surgical instrument with an anti-bacterial coating comprising anti-microbial particles in a autoclavable polymer matrix wherein the anti-microbial particles are positioned to provide an anti-microbial effect at the surface.

10. The method of claim 9 wherein the autoclavable polymer matrix comprises a synthetic polymer having sufficient stability to withstand autoclavable temperatures.

11. The method of claim 9 wherein the autoclavable polymer matrix comprises polyetheretherketone.

12. The method of claim 9 wherein the anti-microbial particles include zeolites, hydroxyapatite and zirconium phosphates.

13. The method of claim 9 wherein the anti-microbial particles are an antibiotic ceramic comprising antibiotic metal ions.

14. The method of claim 13 wherein the antibiotic metal ions comprise silver, copper, zinc, mercury, tin, lead, bismuth, cadmium, chromium and thallium ions.

15. The method of claim 9 wherein the anti-microbial particles comprise inorganic anti-microbial metal salts.

16. The method of claim 9 wherein the anti-bacterial coating has a thickness from about 0.1 mm to about 5.0 mm.
